(19) United States
(12) Patent Application Publication
(10) Pub. No.: US 2025/0031981 A1
(43) Pub. Date: Jan. 30, 2025

(54) A BLOOD PRESSURE DETERMINING SYSTEM AND METHOD THEREOF

(71) Applicant: Turtle Shell Technologies Private Limited, Bengaluru, Kamataka (IN)

(72) Inventors: Parchani GAURAV, Bangalore (IN); Saran VIBHOR, Lucknow (IN); Kaushik PAVAN, Bengaluru (IN); Singh VISHWA, Pune (IN); KS SRIVYSHNAV, Mysore (IN)

(21) Appl. No.: 18/283,069

(22) PCT Filed: Nov. 8, 2022
(86) PCT No.: PCT/IN2022/050978

§ 371 Date: Sep. 20, 2023

(30) Foreign Application Priority Data
Apr. 12, 2022 (IN) 202241021926

(51) Int. Cl. A61B 5/021; A61B 5/00; A61B 5/11; G16H 50/30

(52) U.S. Cl. CPC A61B 5/021; A61B 5/0004; A61B 5/1102; A61B 5/6892; A61B 5/7214; A61B 5/7225; A61B 5/7257; G16H 50/30; A61B 2560/0223

(57) ABSTRACT

The present invention relates to a blood pressure determining system and method thereof utilizing cardiac micro-vibrations. The present system comprises a sensor unit configured to record cardiac micro-vibrations as analog signals and convert the analog signal to micro-voltage digital signals; a processor unit configured to record the micro-voltage digital signals in chronological format and amplify the recorded signals to obtain amplified signals with optimum resolution without loss of information; a computation module configured to: de-noise the amplified signals; generate an energy spectrogram from the denoised amplified signals, said energy spectrogram comprising of a contours trend based on harmonics that align with changes in blood pressure; extract the contours from the harmonics; and scale the contours with a calibration value obtained from a subject, wherein said calibration value is used as a baseline value to obtain the subject's blood pressure values.

### FIELD OF THE INVENTION

The present invention relates to a blood pressure determining system and method thereof that utilizes cardiac micro-vibration signals to obtain an energy spectrogram from which the blood pressure values are obtained. The system and method allow for continuous blood pressure determination and may be contactless.

### BACKGROUND OF THE INVENTION

Blood pressure is a commonly used crucial metric to determine the health of a person. A very high or low blood pressure value can be an indicator of deteriorating health. Monitoring blood pressure is essential to recognize hypertension or hypotension. The current methods of measuring blood pressure (BP) are broadly classified as invasive and non-invasive. Intra-arterial BP measurement is an invasive method of monitoring and measuring BP. The said method is commonly used in the Intensive Care Unit (ICU) and in the operating theatre. This technique involves direct measurement of arterial BP by inserting a cannula needle in a suitable artery. Although the method yields continuous data, it is very risky, as it can introduce infections at the needle entry port and is therefore only used on select ICU patients.

Non-invasive BP measurement and monitoring devices are cuff-based eg. a mercury sphygmomanometer and stethoscope or cuffless being digital devices such as wrist bands, smart watches etc. The cuff-based devices require contact with the person, the cuff has to be accurately placed on the arm, and on occasion needs trained personnel to take the measurements. Moreover, the cuff-based method is not suitable for continuous monitoring as continuous inflation and deflation of the cuff causes discomfort to the patient.

The existing cuffless digital BP measurement and monitoring devices require contact with the person's body to measure and monitor BP, and it has been observed that these devices do not provide consistent results.

The existing cuffless BP measurement devices often use the pulse transit time (PTT) method to calculate BP. This method calculates BP based on the time taken by the pulse to travel between 2 points on the body. The main deficiency of this method is that two sensors are required and the position of the user with respect to these sensors is important for accurate BP measurement. A small shift in the position of the sensors or the user hinders the quality of the measurement. Slight error in alignment can lead to large errors in BP predictions. Also, PTT typically requires two sources of cardiac signals for determining BP, said cardiac signals selected from, including but not limited to, ballistocardiogram, electrocardiogram, phonocardiogram, photoplethysmography, intra-arterial BP waveform, or any other cardiac signals. The time alignment between the multiple sources also becomes critical and is often difficult to achieve. Another disadvantage of having two cardiac signal sources is that the sensors need to have a very high sampling rate, which adds to hardware costs and increases the storage requirements and processing time. Further, requiring a pair of sensors to be placed at different locations on the person's body also adds to user discomfort. Existing cuffless BP measurement techniques, therefore, do not provide a simple, completely automatic and continuous method of BP measurement and monitoring.

Therefore, there is a need for a BP monitoring and measuring system and method that is safe, simple, non-invasive, doesn't require medical practitioner assistance and can continuously provide accurate BP measurements without causing discomfort to the patient.

### SUMMARY OF THE INVENTION

According to one embodiment the present invention is a system for blood pressure determination comprising: a sensor unit configured to record cardiac micro-vibrations as analog signals and convert the analog signal to micro-voltage digital signals; a processor unit configured to record the micro-voltage digital signals in chronological format and amplify the recorded signals to obtain amplified signals with optimum resolution without loss of information; a computation module configured to: de-noise the amplified signals; generate an energy spectrogram from the denoised amplified signals, said energy spectrogram comprising of a contours trend based on harmonics that align with changes in blood pressure; extract the contours from the harmonics; and scale the contours with a calibration value obtained from a subject, wherein said calibration value is used as a baseline value to obtain the subject's blood pressure values.

According to another embodiment the present invention is a method for blood pressure determination comprising: recording cardiac micro-vibrations as analog signals and converting the analog signals to micro-voltage digital signals by a sensor unit; recording the micro-voltage digital signals in chronological order and amplifying said signals to obtain amplified signals with optimum resolution without loss of information by a processor unit; denoising the amplified signals by a computation module; generating, by the computation module, an energy spectrogram from the denoised amplified signals, said energy spectrogram comprising of a contours trend based on harmonics that align with changes in blood pressure; and extracting the contours and scaling said contours with a calibration value obtained from a subject, wherein said calibration value is used as a baseline value, by the computation module, to obtain the subject's blood pressure values.

### BRIEF DESCRIPTION OF THE DRAWINGS

Reference will be made to embodiments of the invention, examples of which may be illustrated in accompanying figures. These figures are intended to be illustrative, not limiting. Although the invention is generally described in context of these embodiments, it should be understood that it is not intended to limit the scope of the invention to these particular embodiments.

FIG. 1 shows the present system and placement of the sensor unit with respect to the subject, according to one embodiment.

FIG. 2 shows instances of body motion artifacts in the amplified signal, according to one embodiment.

FIG. 3 shows an energy spectrogram overlaid with systolic blood pressure measurements recorded using an arterial catheter.

FIG. 4 shows the flow chart of the present method of determining blood pressure.

FIG. 5a shows the predicted versus actual systolic blood pressure from an arterial catheter recording.

FIG. 5b shows the predicted versus actual systolic blood pressure from a cuff-based recording.

### DETAILED DESCRIPTION OF THE INVENTION

In the following detailed description, the embodiments are described in sufficient detail to enable those skilled in the art to practice the invention and it is understood that other embodiments may be utilized and that changes may be made without departing from the scope of the invention. To avoid detail not necessary to enable those skilled in the art to practice the embodiments described herein, the description may omit certain information known to those skilled in the art. Accordingly, the description and figures are to be regarded in an illustrative rather than a restrictive sense, and all such modifications are intended to be included within the scope of the present teachings. It is to be understood that the singular forms “a,” “an,” and “the” include plural referents unless the context clearly dictates otherwise. It should be emphasized that the term “comprises/comprising” when used in this specification is taken to specify the presence of stated features, integers, steps or components but does not preclude the presence or addition of one or more other features, integers, steps, components or groups thereof.

The instant invention intends to address the afore stated technical disadvantages by providing in one embodiment a system for blood pressure determination comprising: a sensor unit configured to record cardiac micro-vibrations as analog signals and convert the analog signal to micro-voltage digital signals; a processor unit configured to record the micro-voltage digital signals in chronological format and amplify the recorded signals to obtain amplified signals with optimum resolution without loss of information; a computation module configured to de-noise the amplified signals; generate an energy spectrogram from the denoised amplified signals, said energy spectrogram comprising of a contours trend based on harmonics that align with changes in blood pressure; extract the contours from the harmonics; and scale the contours with a calibration value obtained from a subject, wherein said calibration value is used as a baseline value to obtain the subject's blood pressure values.

In one embodiment the present invention allows continuous measurement of the BP of a person in a contact-free manner. While most prior art methods aim to make BP measurement cuffless, in one embodiment the present invention is completely contactless or non-invasive. This method does not use cameras or image analysis and does not need the user to be in a particular position, as required in several prior art methods and systems. The device is comfortable to use and reduces the set-up and monitoring burden that comes with the existing methods.

The present invention enables continuous BP measurements from vibration data, particularly the cardiac micro-vibrations, captured from the human body. The present invention uses ballistocardiography (BCG) technology wherein a graphical representation of the motion of contractions in each heartbeat is obtained. In the present invention a sensor unit measures the motion that arises due to the ejection of blood. The sensor unit that captures these vibrations (BCG readings) is placed near the chest of the subject with an optional medium (mattress, bedcover, cushion, etc.) in between the sensor and the subject. In an embodiment of the present invention, the sensor is a single large area sensor or an array of sensors. In another embodiment the sensor is an array of sensors placed in a housing at the subject's end. In an exemplary embodiment of the present invention, the sensor is of a very low thickness, preferably of around 3 mm and has an outer casing for protecting and covering the housing. The outer casing may be a robust and rugged thin cover made of a material, including but not limited to, a mesh, latex, cloth, polymer etc. that firmly holds the array of sensors in a fixed position. The exemplary sensor unit embodiment is capable of being folded and is lightweight. In the various embodiments of the present invention, the sensor unit is used in a non-invasive manner.

In an embodiment of the present system, in operation, the sensor unit is positioned in a contactless manner at the subject's end and is configured to capture cardiac micro vibrations of the subject as analog data signals. The sensor is capable of capturing micro-vibrations received through a medium placed between the subject and the sensor. For example, the micro-vibrations may be captured through a medium ranging from a thin surface to a thick surface such as a 20-inch mattress. The sensor is further capable of capturing micro-vibrations received from the subject without any medium placed between sensor and the subject. The micro-vibrations (BCG readings) captured by the sensor are associated with cardiac signals. The sensor is configured to record cardiac micro-vibrations as analog signals and convert the analog signal to micro-voltage digital signals. The present invention uses a single cardiac signal source (BCG). Thus, the present system is simpler and operates with a much lower sampling rate and avoids the issues caused by time misalignment and sensor placement errors.

In one embodiment, the sensor unit is connected to a processor unit configured, with a computer program, to record the micro-voltage digital signals in chronological format and amplify the recorded signals to obtain amplified signals with optimum resolution without loss of information. In one embodiment of the present system, the recorded micro-voltage digital signals are amplified up to 2500 times, preferably between 15 to 2500 times. The amplification is performed to obtain the optimized signal with maximized resolution without information loss due to clipping. This is crucial for the device to work in any condition with any thickness and construction of the medium between the user and the sensor. This calibration of the amplification happens once when the subject lies down on the device. The sensor unit may be connected to the processor unit via a wired or wireless connection.

In one embodiment the processor unit transmits the amplified signals to a computation module configured, with a computer program, to de-noise the amplified signals; generate an energy spectrogram from the denoised amplified signals, said energy spectrogram comprising of a contours trend based on harmonics that align with changes in blood pressure; extract the contours from the harmonics; and scale the contours with a calibration value obtained from a subject, wherein said calibration value is used as a baseline value to obtain the subject's blood pressure values.

In one embodiment of the present system, the computation module is configured to denoise the amplified signals by filtering the signal to between 0.2 to 40 Hz. The present invention, in one embodiment, may obtain raw data of a measurable frequency of 0 to 1000 Hz, preferably the measurable frequency obtained being between 0 to 125 Hz. In one embodiment the computation module is configured to denoise the amplified signals using a density based scan clustering algorithm or any other algorithm, to remove body motion artifacts.

In one embodiment of the present system, the energy spectrogram is generated using Short Term Fourier Transform (STFT). In one embodiment the calibration value is measured from the subject using a cuff-based device or any other blood pressure measuring device. In one embodiment of the present system, the computation module is configured to scale the harmonics trend contours using the double sigmoid activation method, any other non-linear scaling method or a linear scaling method.

The processor unit may be connected to the computation module wirelessly or through wires. The computation module may be a smartphone, a computer or a remote cloud server. In one embodiment, the processor unit and the computation module are combined in a single processor. In one embodiment of the system, the processor unit comprises a data acquisition unit configured to record the micro-voltage digital signals in chronological order and a conditioning unit configured to amplify the recorded signals to obtain an amplified signal with optimum resolution without loss of information. The data acquisition unit and the conditioning unit may be as separate micro-processors or combined as in the single processor unit.

In one embodiment the present system further comprises a data receiver module for storing the amplified signals, said data receiver module being a smartphone, a computer or a remote cloud server. The data receiver module transmits the amplified signals to the computation module. In one embodiment the present system further comprises a transmission unit comprising a wireless technology module for transferring the amplified signals to the data receiver module or directly to the computation module. The wireless technology module includes but is not limited to Wi-Fi, Bluetooth classic, or Bluetooth low energy.

In one embodiment of the present system it is a method for blood pressure determination comprising: recording cardiac micro-vibrations as analog signals and converting the analog signals to micro-voltage digital signals by a sensor unit; recording the micro-voltage digital signals in chronological order and amplifying said signals to obtain amplified signals with optimum resolution without loss of information by a processor unit; denoising the amplified signals by a computation module; generating, by the computation module, an energy spectrogram from the denoised amplified signals, said energy spectrogram comprising of a contours trend based on harmonics that align with changes in blood pressure; and extracting the contours and scaling said contours with a calibration value obtained from a subject, wherein said calibration value is used as a baseline value, by the computation module, to obtain the subject's blood pressure values. The blood pressure values may be stored in a database. In one embodiment the present system is a method for contactless blood pressure determination.

The technical challenge with prior art signal processing methods when using BCG readings to determine BP is finding the appropriate signal processing features from the BCG readings. There is a lot of research work on extracting and signal processing heart rate and respiratory rate to obtain BP. However, said methods that model BP as a function of other vitals is not the most effective, as there is a lack of clear correlation between BP and the corresponding changes in breathing rate and heart rate.

The present invention utilizes the energy change that occurs with the change in blood pressure to determine the BP values. The physiological changes that occur with the change in blood pressure result in a corresponding change in the energy of the recorded BCG signal. This energy change is obtained from the recorded cardiac micro-vibrations (BCG signal). An energy spectrogram is plotted using the cardiac micro-vibrations (BCG signal data) in order to visualize the power variation with time, for the subject. Quantifying this visible variation gives an accurate estimate of the BP trend which is then scaled appropriately to get continuous BP measurements.

In an embodiment of the present method, the recorded cardiac micro-vibrations are converted to micro-voltage digital signals that are stored in chronological order. The micro-voltage signals are amplified and then denoised by the computation module. In one embodiment of the present method, the recorded micro-voltage digital signals are amplified up to 2500 times, preferably between 15 to 2500 times. In one embodiment of the present method, denoising of the amplified signals involves filtering the signal to between 0.2 to 40 Hz. In one embodiment of the present method, denoising of the amplified signals is performed using a density based scan clustering algorithm or any other clustering program, to remove body motion artifacts.

The denoised amplified signals are used to generate an energy spectrogram comprising of a contours trend based on harmonics that align with changes in blood pressure. In one embodiment of the present method, the energy spectrogram is generated using STFT. The prior art methods such as root mean square and peak difference are not effective in obtaining the correct trend for blood pressure. The trend that the blood pressure follows is most apparent with the STFT method and the trend that the blood pressure follows is well correlated with the extracted contours. Thus, the use of STFT enables generating an energy spectrogram comprising a contours trend based on harmonics that align with changes in blood pressure. An accurate estimate of the blood pressure can be arrived at by extracting the contours from the harmonics. Finally, the extracted contour needs to be scaled to obtain the blood pressure value in mm of Hg. To do this, first a calibration reading is obtained from the patient. For this, before the prediction begins, another device (like a cuff-based monitor) is used to take one systolic and diastolic reading of the patient. This calibration reading is needed because the extracted contour only provides the times where the BP rises and falls. To get the correct prediction, a baseline is needed from where the magnitude of the change can be estimated. This calibration reading acts as the baseline. Next, to estimate the deviation from the calibration value, a method to scale up the contour is required. The scaling may be performed using the double sigmoid activation method, any other non-linear scaling method or a linear scaling method. In a preferred embodiment, scaling is performed using the double sigmoid activation method to obtain the factor that is to be multiplied by the calibration value. The sigmoidal function estimates the percent-change from the calibration BP value using the contour. The final blood pressure is calculated as: Blood Pressure (systolic/diastolic)=(Double Sigmoid (trend)+1)×Calibration (systolic/diastolic).

FIG. 1 shows the present system and placement of the sensor unit with respect to the subject, according to one embodiment. The sensor unit (100) is placed below the mattress (2) with the subject lying on the mattress. The sensor unit (100) records cardiac micro-vibrations as analog signals and convert the analog signal to micro-voltage digital signals which is relayed to the processor unit (101). The processor unit (101) is configured to record the micro-voltage digital signals in chronological format and amplify the recorded signals to obtain amplified signals with optimum resolution without loss of information. The amplified signals are relayed to the computation module (102) which is configured to de-noise the amplified signals; generate an energy spectrogram from the denoised amplified signals, said energy spectrogram comprising of a contours trend based on harmonics that align with changes in blood pressure; extract the contours from the harmonics; and scale the contours with a calibration value obtained from a subject, wherein said calibration value is used as a baseline value to obtain the subject's blood pressure values. In one embodiment of the system, the processor unit comprises a data acquisition unit (101a) configured to record the micro-voltage digital signals in chronological order and a conditioning unit (101b) configured to amplify the recorded signals to obtain an amplified signal with optimum resolution without loss of information. The data acquisition unit (101a) and the conditioning unit (101b) may be as separate micro-processors or combined as in a single processor unit (101). In one embodiment the present system further comprises a data receiver module (104) for storing the amplified signals obtained from the conditioning unit (101b) or the processor unit (101), said data receiver module being a smartphone, a computer or a remote cloud server. The data receiver module transmits the amplified signals to the computation module. In one embodiment the present system further comprises a transmission unit (103) comprising a wireless technology module (not shown) for transferring the amplified signals to the data receiver module (104) or directly to the computation module (102). The wireless technology module includes but is not limited to Wi-Fi, Bluetooth classic, or Bluetooth low energy. The dotted lines represent in FIG. 1 an embodiment of the system with optional units 103 and/or 104. In one embodiment, the processor unit and the computation module may be combined in a single processor.

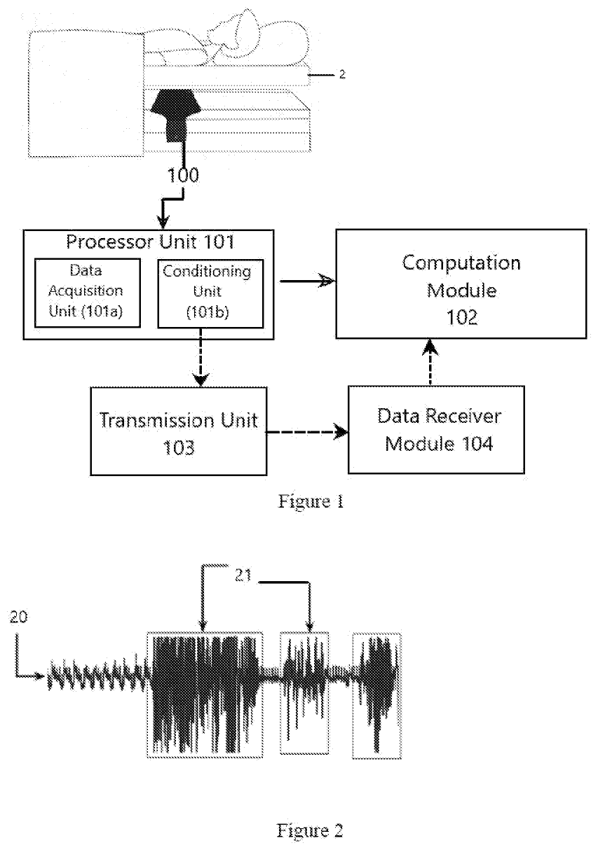

FIG. 2 shows instances of body motion artifacts (21) in the amplified signal (20), according to one embodiment. The amplified signals are subject to denoising to remove noise and artifacts from the signal. To remove the noise, the signal was filtered between 0.2 to 40 Hz. This removes much of the breathing baseline and extraneous noises. There are also sudden bumps (21) in the signal (20) that are caused by motion, snoring, and other similar reasons. These are removed using a density based scan clustering algorithm that determines if a 1-second instance contains such movement instances or body motion artifacts (21).

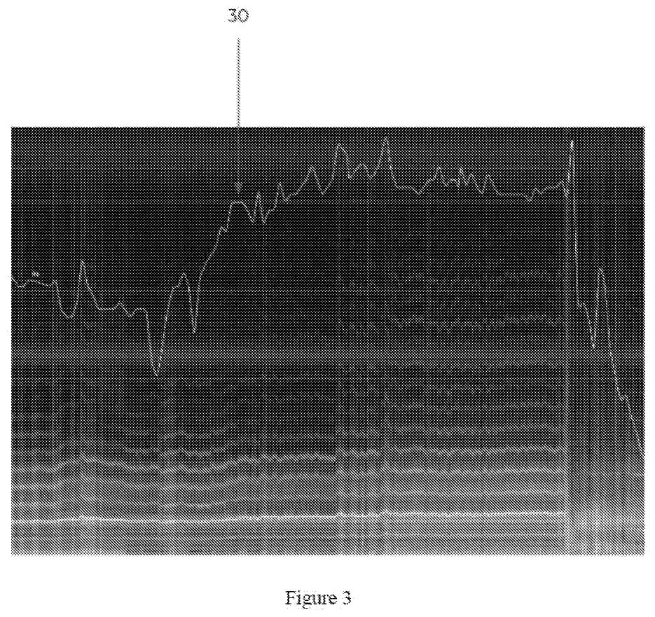

FIG. 3 shows an energy spectrogram overlaid with systolic blood pressure measurements (30) recorded from a patient using an arterial catheter. Visualizing the STFT energy spectrogram as an image, shows the power variation with time for the subject and enables identification of patterns. The spectrogram has 3 dimensions, time on the X axis, frequency on the Y axis, and power represented by the intensity of each pixel on the Z axis. As seen, the grey lines visible in the spectrogram are the harmonics from which the contours are extracted and scaled to arrive at the blood pressure values. The grey lines (each line being for a certain frequency) show a shape pattern that matches the trend shown by the arterial catheter measurements (30). The variation in BP (30), collected simultaneously through the invasive monitoring, matches the variation in the spectrogram of the BCG signal (grey lines). Quantifying this visible variation gives an accurate estimate of the BP trend which can then be scaled appropriately to get the BP measurements.

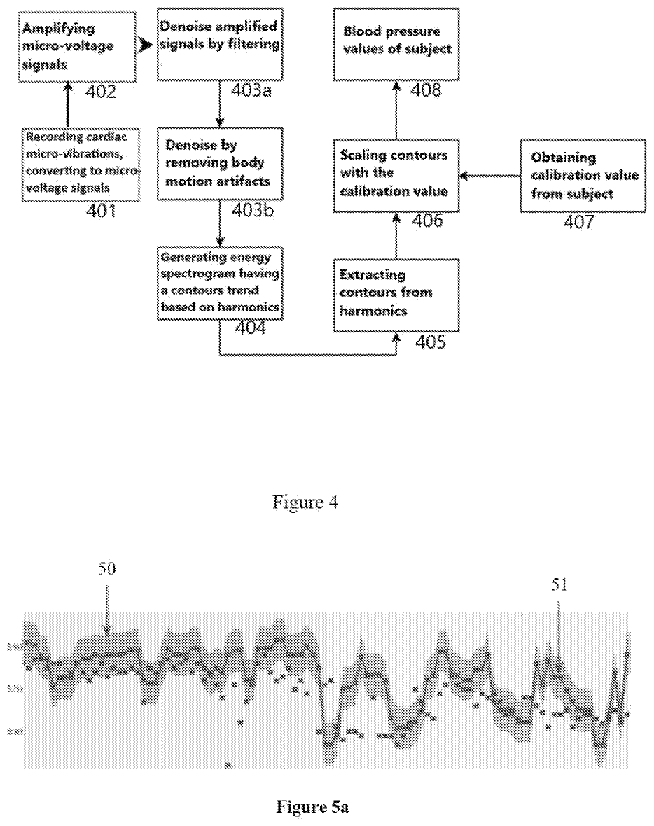

FIG. 4 shows the flow chart of the present method of determining blood pressure. The cardiac micro-vibrations are collected from the subject as analog signals and converted to micro-voltage digital signals (401). In one embodiment this is performed using a sheet of piezoelectric sensors placed under the user while lying down. The sensor captures all the micro-body vibrations arising from cardiac contractions. It also captures the motion arising from breathing, snoring and body movements. Next the micro-voltage digital signals are amplified to obtain amplified signals with optimum resolution without loss of information (402). The amplified signals are then subject to denoising by filtering (403a) and removal of body motion artifacts (403b). Next an energy spectrogram is generated having a contours trend based on harmonics (404). In one embodiment, generating the spectrogram involves taking 10-minute long segments from the denoised amplified signals, and extracting the energy spectrum from them using STFT. The energy spectrogram shows a trend in the harmonics that aligns with the change in BP. This trend in the spectrogram is called the contour. The contours are extracted (405) and scaled (406) using a calibration reading to obtain the subjects BP values (408). The calibration reading is obtained from the subject using another device (eg. a cuff-based monitor) and manually fed into the system (407). The calculated BP values are stored in a database. The database may also be hosted on a standalone smartphone, laptop, tablet, a desktop computer or on a cloud server for remote access. This data can be accessed with proper authorization from anywhere in the world and visualized as graphs or dashboards making it easy to review the information.

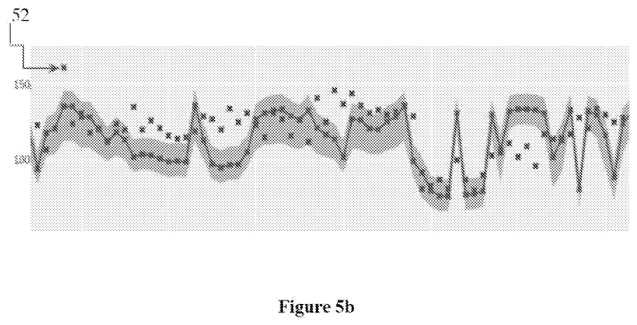

FIG. 5a shows the predicted versus actual systolic blood pressure from an arterial catheter recording. The arterial blood pressure catheter readings were spaced 5 minutes apart and are considered to be more accurate than cuff-based readings. FIG. 5b shows the predicted versus actual systolic blood pressure from a cuff-based recording. A non-invasive cuff-based BP measuring device was used to measure readings at a 30-minute interval. For FIGS. 5a and 5b the solid line (51) represents the predicted blood pressure obtained using the present invention. For FIGS. 5a and 5b the crosses (52) represent the recorded blood pressure readings obtained from the respective devices and the gray area (50) around the solid line (51) is the error margin of 10 mmHg above and below the line. FIG. 5a shows the predicted BP to closely match the BP measurements from the arterial catheter recording. FIG. 5b shows the predicted BP to closely match the BP measurements from the cuff-based recording.

The present system is designed for home or hospital based, onsite or remote, BP monitoring of a subject. The system can be used to convert a bed into a step-down ICU which can monitor a subject's health with minimum intervention. An extremely high or low BP is a sign of deteriorating health. Knowing the BP value can help identify when a patient is getting critical. This paired with other vitals like the heart rate and breathing rate can be used to calculate an early warning score which can raise alerts at the right time.

The present invention is a BP monitoring system and method that is safe, non-invasive, doesn't require medical practitioner assistance and can continuously provide accurate BP measurements without causing discomfort to the patient. The present invention may also be contactless in one embodiment.

While the present invention has been described with respect to certain embodiments, it will be apparent to those skilled in the art that various changes and modifications may be made without departing from the scope of the invention as defined in the following claims.

We claim:

1. A system for blood pressure determination comprising:
a. a sensor unit configured to record cardiac micro-vibrations as analog signals and convert the analog signal to micro-voltage digital signals;
b. a processor unit configured to record the micro-voltage digital signals in chronological format and amplify the recorded signals to obtain amplified signals with optimum resolution without loss of information;
c. a computation module configured to:
de-noise the amplified signals;
generate an energy spectrogram from the denoised amplified signals, said energy spectrogram comprising of a contours trend based on harmonics that align with changes in blood pressure;
extract the contours from the harmonics; and
scale the contours with a calibration value obtained from a subject, wherein said calibration value is used as a baseline value to obtain the subject's blood pressure values.

2. The system as claimed in claim 1, wherein the recorded micro-voltage digital signals are amplified up to 2500 times.

3. The system as claimed in claim 1, wherein the computation module is configured to denoise the amplified signals by filtering the signal to between 0.2 to 40 Hz.

4. The system as claimed in claim 1, wherein the computation module is configured to denoise the amplified signals using a density based scan clustering algorithm or any other algorithm, to remove body motion artifacts.

5. The system as claimed in claim 1, wherein the energy spectrogram is generated using Short Term Fourier Transform.

6. The system as claimed in claim 1, wherein the calibration value is measured from the subject using a cuff-based device or any other blood pressure measuring device.

7. The system as claimed in claim 1, wherein the computation module is configured to scale the harmonics trend contours using the double sigmoid activation method, any other non-linear scaling method or a linear scaling method.

8. The system as claimed in claim 1, wherein the processor unit and the computation module are combined in a single processor.

9. The system as claimed in claim 1, wherein the processor unit comprises a data acquisition unit configured to record the micro-voltage digital signals in chronological order and a conditioning unit configured to amplify the recorded signals to obtain an amplified signal with optimum resolution without loss of information.

10. The system as claimed in claim 1, wherein a data receiver module stores the amplified signals, said data receiver module is a smartphone, a computer or a remote cloud server.

11. The system as claimed in claim 10, wherein a transmission unit comprising a wireless technology module transfers the amplified signals to the data receiver module.

12. The system as claimed in claim 1, wherein the computation module is a smartphone, a computer or a remote cloud server.

13. A method for blood pressure determination comprising:
a. recording cardiac micro-vibrations as analog signals and converting the analog signals to micro-voltage digital signals by a sensor unit;
b. recording the micro-voltage digital signals in chronological order and amplifying said signals to obtain amplified signals with optimum resolution without loss of information by a processor unit;
c. denoising the amplified signals by a computation module;
d. generating, by the computation module, an energy spectrogram from the denoised amplified signals, said energy spectrogram comprising of a contours trend based on harmonics that align with changes in blood pressure; and
e. extracting the contours and scaling said contours with a calibration value obtained from a subject, wherein said calibration value is used as a baseline value, by the computation module, to obtain the subject's blood pressure values.

14. The method as claimed in claim 13, wherein the recorded micro-voltage digital signals are amplified up to 2500 times.

15. The method as claimed in claim 13, wherein denoising of the amplified signals involves filtering the signal to between 0.2 to 40 Hz.

16. The method as claimed in claim 13, wherein denoising of the amplified signals is performed using a density based scan clustering algorithm or any other clustering program, to remove body motion artifacts.

17. The method as claimed in claim 13, wherein the energy spectrogram is generated using Short Term Fourier Transform.

18. The method as claimed in claim 13, wherein the calibration value is obtained using a cuff-based device or any other blood pressure measuring device.

19. The method as claimed in claim 13, wherein the scaling is performed using the double sigmoid activation method, any other non-linear scaling method or a linear scaling method.
